(19) United States
(12) Patent Application Publication
(10) Pub. No.: US 2004/0202673 A1
(43) Pub. Date: Oct. 14, 2004

(54) Constructs of branched synthetic peptide immunogens with artificial T helper cell epitopes coupled to B cell epitopes

(75) Inventor: Jen-Pin Huang, Taipei Hsien (TW)

Correspondence Address: FINNEGAN,HENDERSON,FARABOW,, GARRETT & DUNNER, LLP., 1300 I STREET, N.W., WASHINGTON, DC, 20005-3315, US

(73) Assignees: Jen-Pin Huang, Taipei Hsien (TW); Genesis Biotech Inc.

(21) Appl. No.: 10/411,208

(22) Filed: Apr. 8, 2003

(51) Int. Cl. A61K 39/00; C07K 14/47

(52) U.S. Cl. 424/185.1; 530/404

(57) ABSTRACT

The present invention relates to the construction of synthetic peptide immunogens to induce the production of anitbodies specific to a designated B epitope, usually a self molecule. The peptide immunogens are synthesized in branched forms with artificial Th epitopes conjugated, directly or through a spacer, to a B epitope in a specific orientation. The novel peptide immunogens are designed to elicit high level of antibodies for immunotherapy or immunomodulation of the body regulatory processes.

### BACKGROUND OF THE INVENTION

1. Field of Invention

This invention relates generally to the construction of carrier free synthetic peptide immunogens and more particularly to the construction of different types of novel multiple antigen peptide systems containing monomers of a specific T helper cell epitope (Th epitope) linked to a B cell epitope (B epitope) effective in eliciting immune responses against the B epitope. These novel multiple antigen peptide systems are ideal vaccine candidates in immunotherapy and immunomodulation of body functions.

2. Description of Related Art

The immune system of vertebrates protects the organism from antigenic invaders in many ways. In general, the immune response can be classified into two classes, i.e. the (1) humoral response involving B lymphocytes and antibodies secreted by B cells and (2) the cell-mediated response involving a class of T lymphocytes called cytotoxic (killer) T cells. The humoral response is mediated by the antibodies recognizing and binding to the antigen and thereby triggering other components of the immune system to destroy the antigen. The entire mechanism is first initiated by an antigen being processed by an antigen presenting cells which proteolytically process the antigens to generate peptide fragments which are subsequently exposed on the cell surface in complex with MHC class II molecules. B cells can act as highly potent antigen presenting cells when they endocytose antigens via their specific surface immunoglobulin receptors. The subsequent recognition of T helper (Th) epitope on the peptides presented by MHC class II on the surface of B cells by T cell receptor (TCR) leads to direct T-cell help to the B cell and eventually to generation of antibodies against the intact antigen. It is likely that TCR's binding to the MHC/peptide antigen complex initiates the signal transduction as dimerization or oligomerization of cell surface receptors upon binding of ligand is a common mechanism that initiates signal transduction. T helper cells provide the signals to B cells to enable them to secrete antibodies specific to the antigen.

The immune system's ability to generate molecules capable of highly specific molecular recognition and application of the approach to any molecular target has made antibody technology a powerful tool in the treatment of diseases and immunomodulations of the body's regulatory processes. The method, however, suffers from the disadvantage that when the antigen is “self” it is poorly immunogenic. This has been well illustrated by the long-standing experience with immunocastration vaccines containing the Gonadotropin Releasing Hormone (GnRH). GnRH plays a key role in reproduction by regulating gonadotropin secretion. It is produced in the hypothalamus and conveyed to the anterior pituitary gland via a specialized system of blood vessels. It is responsible for selectively causing the release of follicle stimulating hormone (FSH) and luteinizing hormone (LH) from the anterior pituitary gland. FSH and LH control spermatogenesis, ovulation, and estrus and ultimately direct the secretion of male hormones—androgen and testosterone—and female hormones—estrogen and progesterone. Active immunization against GnRH is known to reduce these sexual hormones and can be used as a mean of reversible contraception. Also, inhibition of GnRH, and thus inhibiting the sexual hormones, can be a therapeutic mean to treat hormone dependent illnesses such as prostate cancer, breast cancer, and endometriosis, estrogen mediated development of endometrial tissue outside the uterus. Furthermore, GnRH may be used in the immunocastration of male animals to prevent boar taint, an offensive aroma or taste found in un-castrated male animal food resulting from a high level of androstenone, which is usually eliminated by mechanical castration, which, however, will lessen growth performance of the castrated animal. Thus, immunocastration has the advantage of eliminating boar taint while preserving the normal growth performance.

Not all animals vaccinated with immunocastration vaccines, however, are equally affected, even after multiple vaccinations. Immunocastration vaccines are acceptable as an alternative of surgical castration only if all animals are equally affected using a minimum of vaccinations. Vaccines based on the GnRH peptide itself cannot meet the goal as no effective amount of antibodies is induced against this self molecule.

Attempts have been made to induce antibodies against self molecules. It has been reported that the antigen-specific B cells can be triggered to produce high titers of high-affinity neutralizing IgG antibodies, the type of antibodies found in many autoimmune diseases such as myasthenia gravis, lupus erythematosus, rheumatoid arthritis etc., in mice where the mice were immunized with antigen covalently coupled to a carrier protein to provide new T helper epitopes linked to the B epitope. Therefore, a small peptide containing a B epitope, a weak immunogen when administered alone, is usually coupled to an extraneous protein carrier, a source of determinants, to stimulate T helper cells. However, there are problems associated with the use of carrier proteins i.e. 1) chemical coupling of the peptide to the carrier protein may cause changes to the determinants of interests and the random reactions will result in a heterogeneous preparation in size and composition, 2) carrier protein may introduce undesirable immune response, and 3) the peptide-carrier conjugate may elicit irrelevant immune response misdirected to the carrier protein instead of the target site. To avoid these problems, Th epitopes have been used to replace the carrier proteins to elicit T helper cell response. A Th epitope may be connected in tandem with a B epitope as a synthetic peptide construct. In the alternative, the synthesis of multiple antigenic peptide (MAP) in which multiple copies of the same peptide, Th epitope linked to B epitope, are assembled on the lysine core through the α and ε amino groups has been described (Fitzmaurice et al., in The assembly and immunological properties of non-linear synthetic immunogens containing T-cell and B-cell determinants, vol 14, Vaccine 1996, pp553-560).

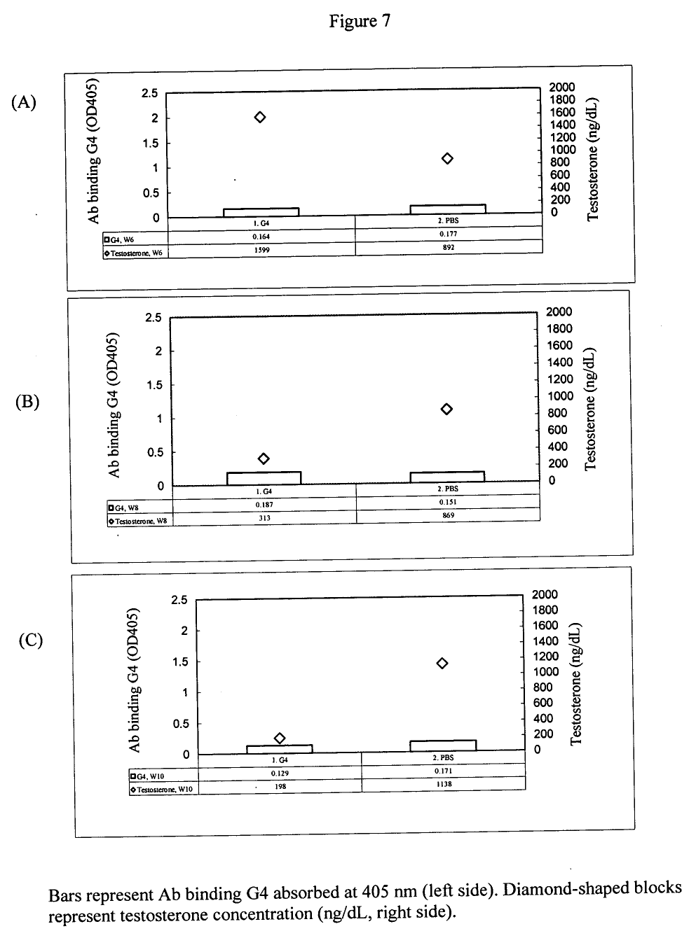

It has been postulated that in general, branched immunogens with multiple copies of the B epitope are superior immunogens (Fitzmaurice et al., in The assembly and immunological properties of non-linear synthetic immunogens containing T-cell and B-cell determinants, vol 14, Vaccine 1996, pp553-560). However, there are exceptions where branched determinants did not elicit detectable antibody level in CBA Mice as did their tandem counterpart, where the B cell determinant is determined by the sequence TLKLATG and the T cell determinant is represented by either ALNNRFQIKGVELKS or PKYVKQNTLKLA (Fitzmaurice et al., in The assembly and immunological properties of non-linear synthetic immunogens containing T-cell and B-cell determinants, vol 14, Vaccine 1996, pp553-560). FIG. 7 shows an experiment result where branched form of GnRH B epitope failed to elicit an immune response in mice. Also, in assembling designer immunogens, the choice of Th epitope plays a role in the effectiveness of the synthetic immunogen. Artificial Th epitopes, modeled after known naturally occurring Th epitopes by shortening, adding, and/or modifying the known Th epitopes, have been employed in experiments to couple to usually weak immunogens to elicit effective immune response.

### BRIEF SUMMARY OF THE INVENTION

The present invention is directed to different types of novel non-linear, branched, synthetic peptide immunogen constructs. Each construct employs different artificial Th epitopes. The Th eipitope is connected to a B epitope in MAP form to induce antibodies directed to the B epitope connected thereto. These novel peptide immunogen constructs elicited high titers of antibodies targeted specifically to the B epitope and produced measurable bioefficacy results. The composition of the present invention may be used to prepare vaccines for immune protection from infectious diseases, immunotherapies to treat disorders resulting from malfunction of normal physiological processes, immunotherapies for the treatment of cancer, and intervention in and modification of normal physiological processes.

### BRIEF DESCRIPTION OF DRAWINGS

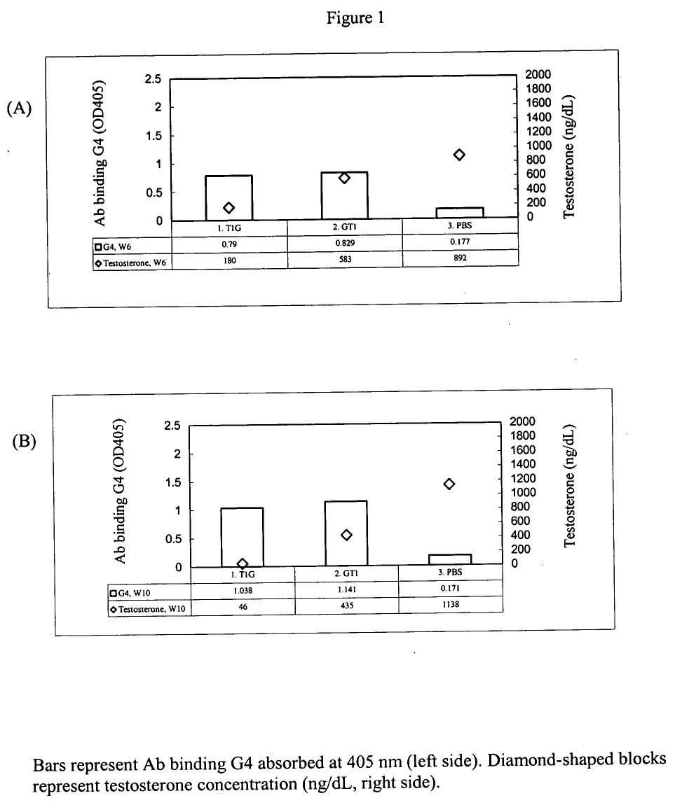

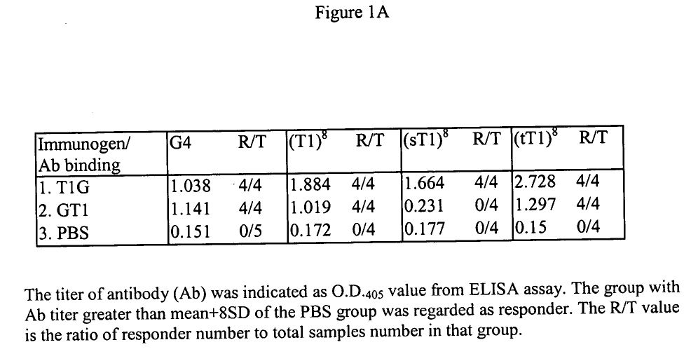

1. FIG. 1 shows amount of antibodies raised against G4 and the corresponding serum testosterone level in Male BALB/c mice immunized with T1G and GT1.

2. FIG. 1A shows the fine specificity of antibodies raised in binding to G4, (T1)⁸, (sT1)⁸, and (tT1)⁸.

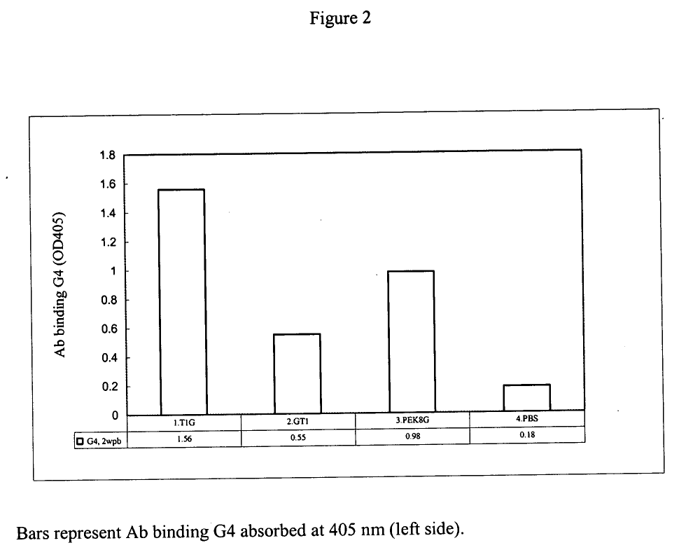

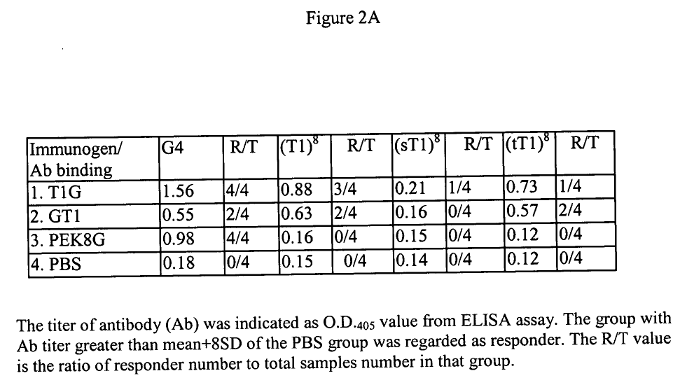

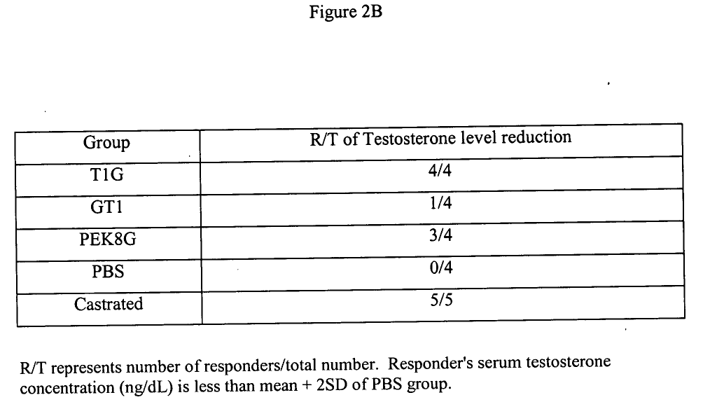

3. FIG. 2 shows amount of antibodies raised against G4 in male rats immunized with T1G and GT1.

4. FIG. 2A shows the fine specificity of antibodies raised in binding to G4,(T1)⁸, (sT1)⁸, and (tT1)⁸.

5. FIG. 2B shows the serum testosterone level in male rats immunized with T1G, GT1, and PEK8G.

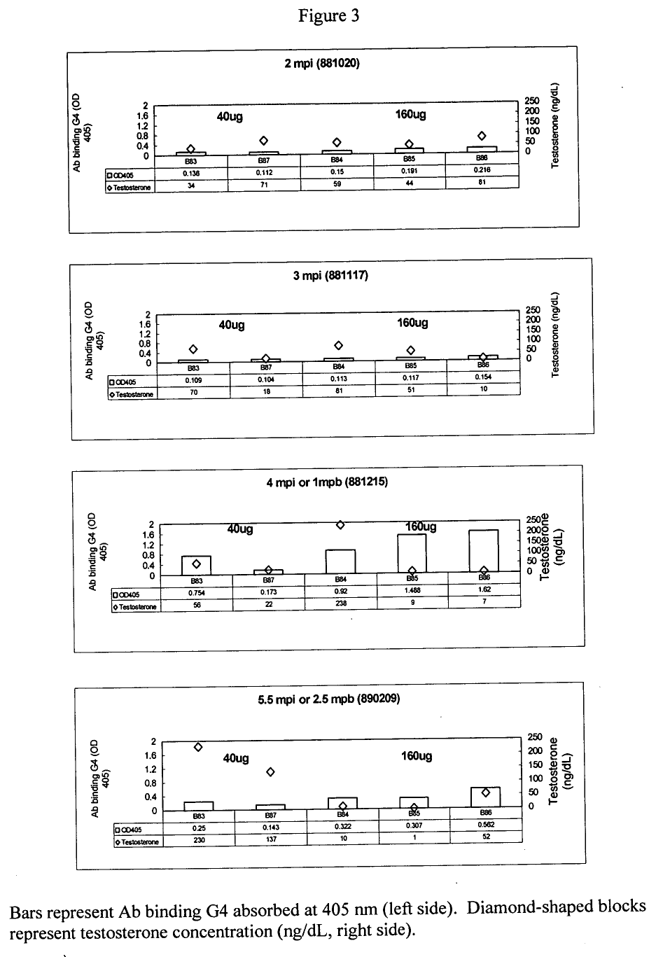

6. FIG. 3 shows the amount of antibodies raised against G4 and serum testosterone level in juvenile beagles immunized with T1G.

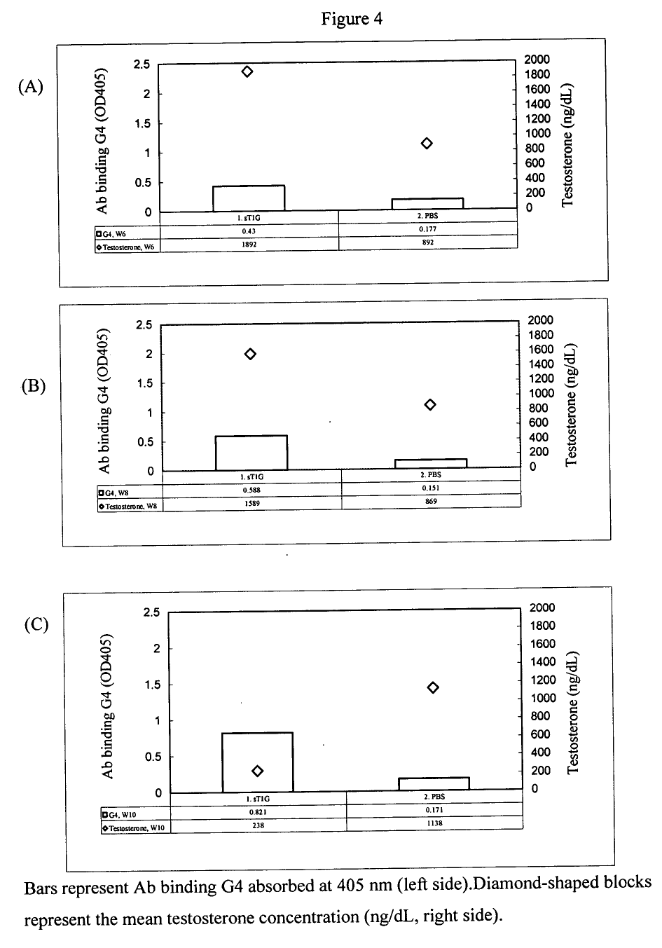

7. FIG. 4 shows the amount of antibodies raised against G4 and serum testosterone level in male BALB/c mice immunized with sT1G.

8. FIG. 4A shows the fine specificity of antibodies raised in binding to G4,(T1)⁸, (sT1)⁸, and (tT1)⁸ in male BALB/c mice immunized with sT1G.

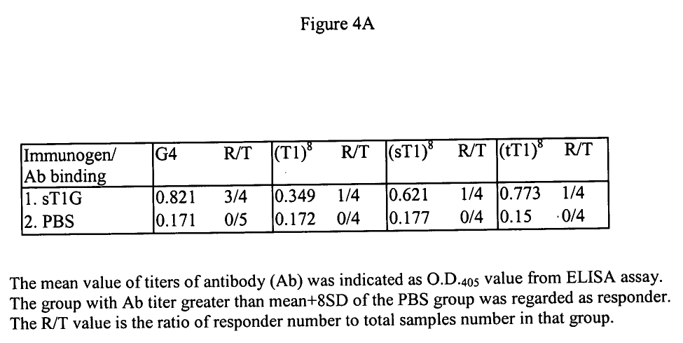

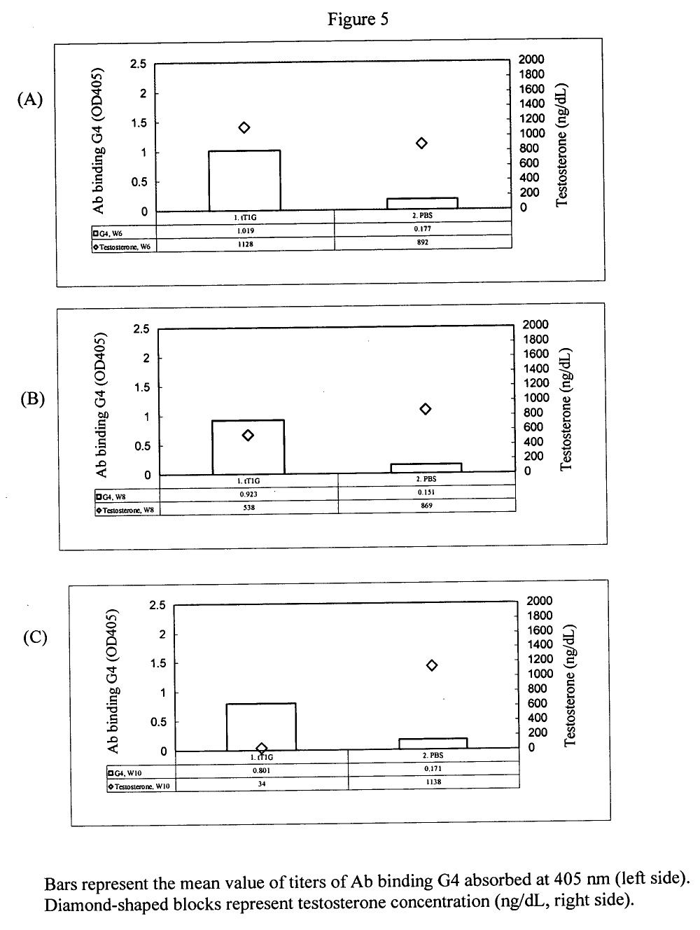

9. FIG. 5 shows amount of antibodies raised against G4 and serum testosterone level in male BALB/c mice immunized with tT1G.

10. FIG. 5A shows the fine specificity of antibodies raised in binding to G4,(T1)⁸, (sT1)⁸, and (tT1)⁸ in male BALB/c mice immunized with tT1G.

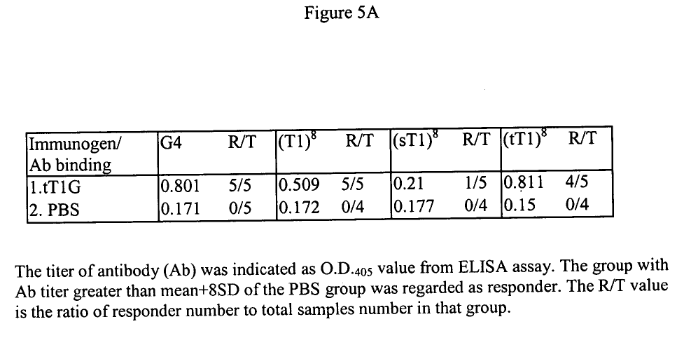

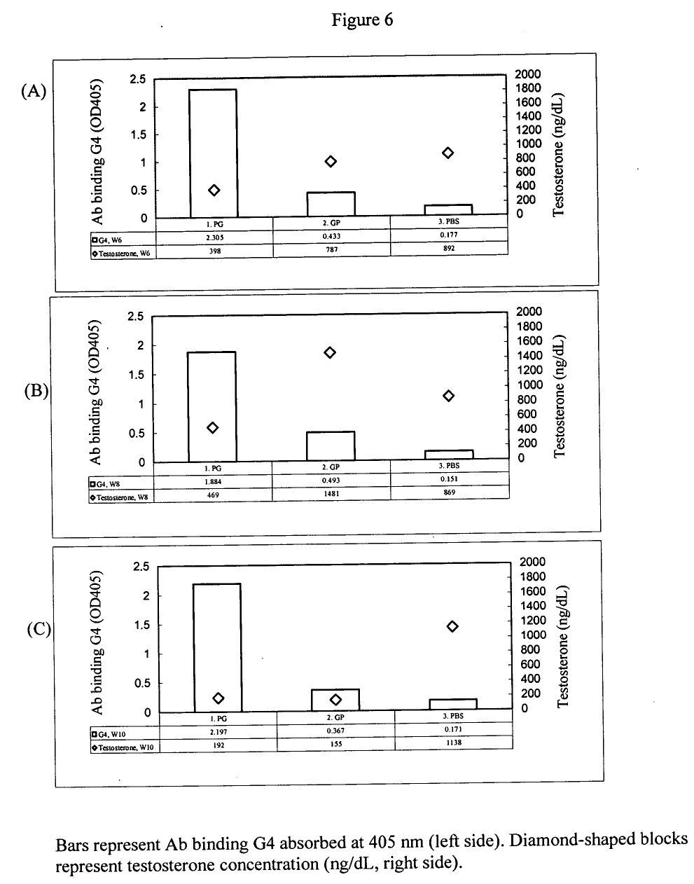

11. FIG. 6 shows amount of antibodies raised against G4 and serum testosterone level in male BALB/c mice immunized with PG and GP.

12. FIG. 7 shows amount of antibodies raised against G4 and serum testosterone level in male BALB/c mice immunized with G4.

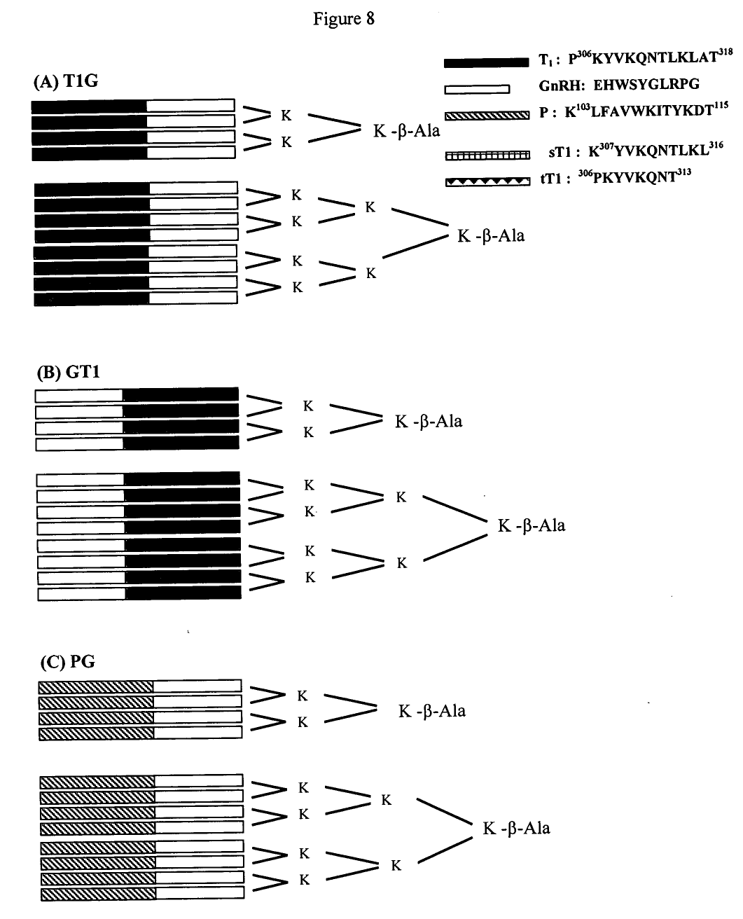

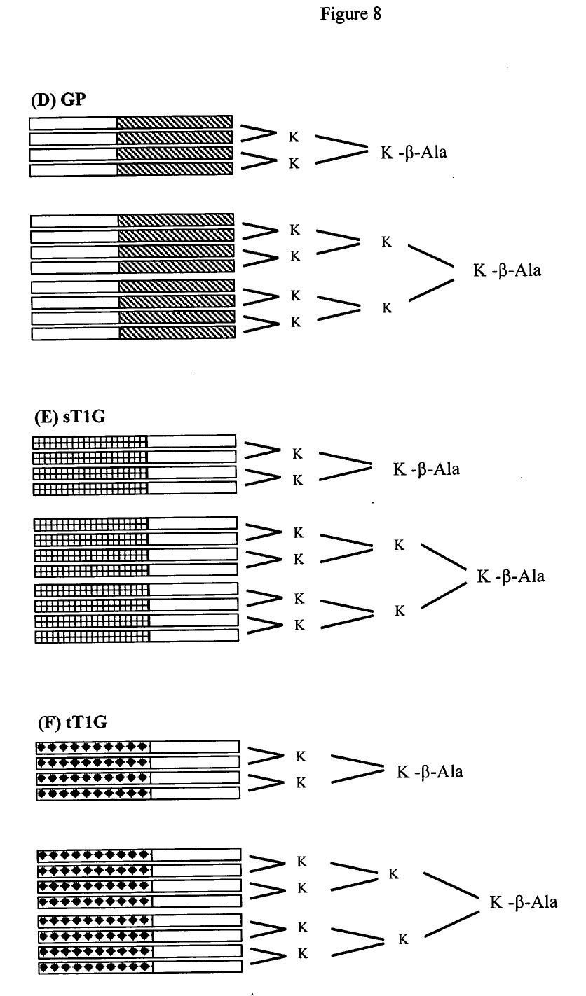

13. FIG. 8 is a schematic representation of the MAP constructs containing 4 and 8 monomers linked through lysine core. Each monomer comprises one copy of GnRH and/or one copy of T cell epitope derived from influenza virus haemagglutinin heavy chain (T1, sT1, and tT1) or poliovirus type I VP1 protein (P).

### DETAILED DESCRIPTION OF THE INVENTION

The present invention relates to different novel peptide immunogen constructs. In each construct, a Th epitope connected to a B epitope (e.g., an immunosilent epitope) is covalently linked to a branching dendritic core which is composed of one or more bifunctional units e.g., lysine, cysteine, aspartic acid, glutamic acid, and ornithine, and different Th epitopes are used for each type of construct. The term “peptide immunogen” as used herein refers to the branched chimeric Th epitope/B epitope peptide. By linking a B epitope to the artificial Th epitope, an immune response directed to the B epitope is induced. Thus, the peptide immunogen can be a useful tool in eliciting antibodies against self molecules for purposes of immunotherapy and immunomodulation of body regulatory processes.

In some embodiments, the Th epitope is derived from the heavy chain of influenza virus hemagglutinin protein. For example, SEQ ID NO 1(T1), can be used to elicit antibody response to the B epitope connected thereto in a MAP form. In the peptide immunogen, SEQ ID NO 1 is covalently linked to the amino end (N terminus) or carboxyl end (C terminus) of the B epitope via conventional peptide bonds, forming a monomer subunit. In each monomer, the Th epitope can be connected to the B epitope directly or through a spacer, typically comprising one or more amino acid residues such as Glycine. The spacer physically separates the Th epitope and B epitope for better binding by the TCR. The spacer also disrupts any artificial secondary structure formed by the tandem Th epitope/B epitope and thereby eliminates possible interference with T helper cell and B cell responses. Four monomer or eight monomer subunits can be distributed as a dentritic arms on a dendritic core matrix in MAP form, see FIG. 8. The synthesis of tetrameric and octameric MAP peptides are accomplished manually by a stepwise solid-phase procedure with Fmoc strategy, e.g., on

[Fmoc-Lys(Fmoc)]2-Lys-βAla-Wang resin (0.77 mmol/g, ACT, Louisville, Ky., USA) and Fmoc strategy on [Fmoc-Lys(Fmoc)]4-Lys2-Lys-βAla-Wang resin (0.77 mmol/g, ACT, Louisville, Ky., USA) respectively. The coupling of Fmoc amino acids is performed in N-methylpyrrolidone using the dicyclohexylcarbodiimide/hydroxybenzotriazole procedure. After completion of the MAP synthesis, deprotection and cleavage from resin support is accomplished by treatment with trifluoroacetic acid. The final products are characterized by reverse phase high performance liquid chromatography and MALDI TOF Mass Spectrum.

As an example, SEQ ID NO 1 can be connected to gonadotropin releasing hormones (GnRH), SEQ ID NO 5, a ten amino acids peptide, via a peptide bond, directly at the N terminus (T1G) or the C terminus (GT1) forming a monomer. Connection at the N terminus is preferred. Four monomers are then polymerized into tetrameric MAP form. Both T1G and GT1 in tetrameric MAP form induced high titers of antibodies against GnRH. The peptide immunogen with the preferred connection at the N terminus is found to reduce testosterone level in male animals.

Another peptide immunogen construct utilizes a Th epitope derived from the shortened heavy chain of influenza virus hemagglutinin protein, SEQ ID NO 2 (sT1). SEQ ID NO 2 can be covalently linked to the B epitope via a peptide bond either directly or through a spacer as described above. Copies of SEQ ID NO 2 connected to the B epitope can be polymerized into tertrameric or octameric MAP form, see FIG. 8, using a stepwise solid-phase procedure with Fmoc strategy on

[Fmoc-Lys(Fmoc)]2-Lys-βAla-Wang resin (0.77 mmol/g, ACT, Louisville, Ky., USA) or Fmoc strategy on [Fmoc-Lys(Fmoc)]4-Lys2-Lys-βAla-Wang resin (0.77 mmol/g, ACT, Louisville, Ky., USA) respectively. The coupling of Fmoc amino acids was performed in N-methylpyrrolidone using the dicyclohexylcarbodiimide/hydroxybenzotriazole procedure. After completion of the MAP peptide synthesis, deprotection and cleavage from resin support is accomplished by treatment with trifluoroacetic acid. The final products were characterized by reverse phase high performance liquid chromatography and MALDI TOF Mass Spectrum.

For example, SEQ ID NO 2 is connected to GnRH B epitope directly, via peptide bond linkage, at its N terminus (sT1G). The peptide immunogen is polymerized in tetrameric MAP form. It is able to induce an immune response against the GnRH B epitope.

Artificial Th epitope derived from a truncated heavy chain of influenza virus hemagglutinin protein, SEQ ID NO 3 (tT1), can also elicit an immune response against the B epitope connected thereto. SEQ ID NO 3 can be connected to the B epitope directly or through a spacer as described above. The peptide immunogen can by polymerized in tetrameric or octameric MAP forms, see FIG. 8. The tetrameric and octameric MAP forms are synthesized through a stepwise solid-phase procedure with Fmoc strategy on [Fmoc-Lys(Fmoc)]2-Lys-βAla-Wang resin (0.77 mmol/g, ACT, Louisville, Ky., USA) and Fmoc strategy on

[Fmoc-Lys(Fmoc)]4-Lys2-Lys-βAla-Wang resin (0.77 mmol/g, ACT, Louisville, Ky., USA) respectively. The coupling of Fmoc amino acids was performed in N-methylpyrrolidone using the dicyclohexylcarbodiimide/hydroxybenzotriazole procedure. After completion of the MAP peptide synthesis, deprotection and cleavage from resin support is accomplished by treatment with trifluoroacetic acid. The final products were characterized by reverse phase high performance liquid chromatography and MALDI TOF Mass Spectrum.

For example, SEQ ID NO 3 is connected to the GnRH B epitope directly at its N terminus via peptide bond linkage (tT1G), and the peptide immunogen is polymerized in tetrameric MAP form. It is able to induce immune response against the B epitope.

In another peptide immunogen construct of the invention, Th epitope derived from poliovirus type I VP1 Protein, SEQ ID NO 4 (P), can be connected to the N terminus or C terminus of a B epitope to elicit immune response. SEQ ID NO 4 can be linked to the B epitope directly or through a spacer at the N or C terminus of the B epitope. The peptide immunogen construct can take on the form of a tetrameric or octameric MAP polymer, see FIG. 8. The tetrameric or octameric MAP polymers are synthesized via a stepwise solid-phase procedure with Fmoc strategy on

[Fmoc-Lys(Fmoc)]2-Lys-βAla-Wang resin (0.77 mmol/g, ACT, Louisville, Ky., USA) or Fmoc strategy on [Fmoc-Lys(Fmoc)]4-Lys2-Lys-βAla-Wang resin (0.77 mmol/g, ACT, Louisville, Ky., USA) respectively. The coupling of Fmoc amino acids can be performed in N-methylpyrrolidone using the dicyclohexylcarbodiimide/hydroxybenzotriazole procedure. After completion of the MAP peptide synthesis, deprotection and cleavage from resin support is accomplished by treatment with trifluoroacetic acid. The final products are characterized by reverse phase high performance liquid chromatography and MALDI TOF Mass Spectrum.

As an example, SEQ ID NO 4 is connected to the GnRH B epitope at its N terminus (PG) in one experiment and its C terminus (GP) in another experiment. In both cases, SEQ ID NO 4 is directly linked to the GnRH B epitope directly via a peptide bond, and the peptide immunogens are polymerized in tertrameric MAP form.

Since Th epitopes can consist of continuous or discontinuous amino acid segments, not every amino acid segment of the Th epitope is necessarily involved in the MHC recognition. The above described Th epitopes would include immunologically functional homologs, including immune enhancing homologs, crossreactive homologs, conservative substitutions, additions, deletions and insertions, segments of the Th epitopes, and sequences that are at least 50%, 60%, 70%, 75%, 80%, 85%, 90%, or 95% homologous (identical) to the Th epitopes sequences described above. As used herein, percent homology or identity of two amino acid or nucleic acid sequences is determined using the algorithm of Karlin and Altschul (Proc. Natl. Acad. Sci. USA 87:2264-2268, 1990), modified as in Karlin and Altschul (Proc. Natl. Acad. Sci. USA 90-5873-5877, 1993). Such algorithm is incorporated into the XBLAST programs of Altschul et al. (J. Mol. Biol. 215:403-410, 1990). BLAST protein searches are performed with the XBLAST program, score=50, word length=3, to obtain amino acid sequences homologous to the reference polypeptide. When using the XBALST program, the default parameters of said program is used.

The present invention also relates to a delivery system comprising a composition of an immunologically effective amount of the peptide immunogens and a pharmaceutically acceptable carrier. A suitable dosage of the peptide immunogens generally contains about 0.005 mg to about 1.5 mg of the peptide immunogen per kg body weight. This dosage can be divided into an appropriate amount per dose when delivered in multiple doses. Dosage will depend on age, body weight, and health conditions of the subject as is well known in the vaccine and therapeutic arts. The suitable amount of peptide immunogens can be formulated in adjuvants, emulsifiers, or any other pharmaceutically acceptable carriers in vaccine compositions such as alum, incomplete Freund's adjuvant and ISA 206 (Montanide). The formulation can be readily determined by one of ordinary skill in the art, including formulations for immediate and/or sustained release. The formulation may be administered by any convenient route, including subcutaneous, oral, intramuscular, intraperitoneal, or other parenteral or enteral route.

As a specific example, the present invention provides for a method to induce anti GnRH antibodies to reduce testosterone to a level achieving effective contraception in mice by administering a pharmaceutical composition comprising the peptide immunogens containing GnRH to a mammal for an appropriate time. The appropriate dosage is about 1.428 mg of peptide immunogen per kg body weight.

### EXAMPLE 1

A. Peptide Synthesis. The peptide immunogens comprising Th epitope of SEQ ID NO 1 connected to the N terminus and C terminus of the GnRH B epitope were synthesized in tetrameric MAP form. The synthesis of tetrameric MAP peptides were accomplished manually by a stepwise solid-phase procedure with Fmoc strategy on [Fmoc-Lys(Fmoc)]2-Lys-βAla-Wang resin (0.77 mmol/g, ACT, Louisville, Ky., USA). The coupling of Fmoc amino acids was performed in N-methylpyrrolidone using the dicyclohexylcarbodiimide/hydroxybenzotriazole procedure. After completion of the MAP peptide synthesis, deprotection and cleavage from resin support is accomplished by treatment with trifluoroacetic acid. The final products were characterized by reverse phase high performance liquid chromatography and MALDI TOF Mass Spectrum.

B. Immunization Protocol. Three groups of five male BALB/c mice were used at 4-5 weeks old, with one group as a control group. They were bred under specific pathogen-free conditions and transferred to a conventional animal house for the experiment. All aspects of the work including housing, experimentation and disposal of animals were performed in general according to the International Guiding Principles for Biomedical Research Involving Animals (CIOMS Publication No. ISBN 92 90360194, 1985). First, the mice were weighed. The average weight of male BALB/c mice at 4-5 weeks old is 35 gram. Second, for group 1, 50 μg of the immunogen peptides (tetrameric MAP peptide with SEQ ID NO 1 connected to the N terminus of GnRH B epitope) is solubilized in 100 μl PBS, emulsified with an equal volume of adjuvant ISA 206 (Montanide), and mixed by POLYTRON PT3100 (kinematic model) at 2,000 rpm for 1 hour. For group 2, 50 μg of the immunogen peptides (tetrameric MAP peptide with SEQ ID NO 1 connected to the C terminus of GnRH B epitope) is solubilized in 100 μl PBS, emulsified in 100 μl ISA 206 (Montanide), and mixed by POLYTRON PT3100 (kinematic model) at 2,000 rpm for 1 hour. For the control group, adjuvant plus PBS was used. Third, on week 0, each of the mice in the first and second groups was then inoculated with the respective peptide immunogen at a dosage of 50 μg/200 μl subcutaneously. Mice in the control group were injected with 200 μl containing PBS and adjuvant (1:1). Booster injections with same inoculations were given subcutaneously on weeks 2 and 4. Fourth, blood was collected by retroorbital plexus puncture on the 6th and 10th week for ELISA assay. The blood was centrifuged at 5,000 r.p.m. for 10 minutes and sera were stored at −20° C. in a freezer.

C. Immunogenicity Determination. Serum samples were assayed for antibodies against various peptides of G4 (tetrameric MAP form of GnRH), T1in octameric MAP form (T1)₈, sT1 in octameric MAP form (sT1)₈, and tT1 in octamreic MAP form (tT1)₈ by ELISA. ELISA assays were performed using 96-well ELISA plates (Nalge nunc). The various peptide antigens were adsorbed to the plates, at a concentration of 0.5 μg/well in 100 μl/well of bicarbonate coating buffer (1.378 g Na₂CO₃, 2.94 g NaHCO₃ in 1 L ddH₂O) and incubated overnight at 4° C. Then, the coating buffer was discarded and, ELISA plates were washed three times with wash buffer (0.5 ml Tween-20 in one litter of 1×PBS). Then the plates were blocked with 5% BSA at 100 μl per well and incubated over night at 4° C. Blocking solution was discarded and the plates were stored at −20° C. until use. Test sera were diluted 1:100× in 5% BSA and placed at 100 μl per well. Test sera were allowed to react at room temperature for 2 hours. Test sera dilutions were then discarded and plates washed three times with wash buffer. Then goat-anti-mouse IgG (Sigma, Fab specific, A-1293, Lot 28H4859, Alkaline phosphatase conjugate) at 1:5000× dilution were placed at 100 μl per well and allowed to react at room temperature for 2 hours. Sera dilution was then discarded, and the plates were washed three times with wash buffer. 100 μl/well of color developing buffer (15 mg pNPP (p-Nitrophenyl Phosphate) (3 tablets of product No. 34047 of Pierce) in 15 ml of 10 mM Diethanolamine buffer (PH 9.5)) were added to the plates and allowed to develop for half an hour at 37° C. The absorbances, optical density, were measured at 405 nm.

D. Immunogen Bioefficacy Determination. Testosterone levels of the three groups of mice were determined on 6 wpi and 10 wpi with the Ciba Corning Automated Chemiluminescence (ACS™) Testosterone assay kit. The ACS Testosterone assay measures testosterone concentration up to 1500 ng/dL with a minimum detectable concentration of 10 ng/dL (=0.347 nmol/L). Serum testosterone levels below 10 ng/dL was considered “castration” levels, however, below 57.6 ng/dL (2 nmol/L) was considered as a responder of the immunocontraception vaccine studied.

E. Results. As shown in FIG. 1, on week 10 post immunization, both peptide immunogens T1G and GT1 raised high antibody response against G4, however, GT1 has a stronger antibody response against G4. In terms of the fine specificity of antibodies raised, see FIG. 2, T1G also produced high antibody response to (T1)₈ and its derivatives (sT1)⁸ and (tT1)⁸ than GT1. With respect to testosterone reduction, T1G has a significant result in reducing testosterone level in male mice with a reading of 46 ng/dL as shown in FIG. 1.

### EXAMPLE 2

A. Peptide Synthesis. The peptide immunogens comprising Th epitope of SEQ ID NO 1 connected to the N terminus and C terminus of the GnRH B epitopeis are synthesized in tetrameric MAP form. The synthesis of tetrameric MAP peptides were accomplished manually by a stepwise solid-phase procedure with Fmoc strategy on [Fmoc-Lys(Fmoc)]2-Lys-βAla-Wang resin (0.77 mmol/g, ACT, Louisville, Ky., USA). The coupling of Fmoc amino acids was performed in N-methylpyrrolidone using the dicyclohexylcarbodiimide/hydroxybenzotriazole procedure. After completion of the MAP peptide synthesis, deprotection and cleavage from resin support is accomplished by treatment with trifluoroacetic acid. The final products were characterized by reverse phase high performance liquid chromatography and MALDI TOF Mass Spectrum.

B. Immunization Protocol. Five groups of male rats were used at 4-5 weeks old, with three groups as a control group. All groups contain four rats, except group five contains five castrated rats. They were bred under specific pathogen free conditions and transferred to a conventional animal house for the experiment. All aspects of the work including housing, experimentation and disposal of animals were performed in general according to the International Guiding Principles for Biomedical Research Involving Animals (CIOMS Publication No. ISBN 92 90360194, 1985). First, the rats were weighed. The average weight of male rats at 4-5 weeks old is 100 grams. Second, for group 1, each 100 μg of the immunogen peptides (tetrameric MAP peptide with SEQ ID NO 1 connected to the N terminus of GnRH B epitope) is solubilized in 200 μl PBS, emulsified with an equal volume of adjuvant ISA 206 (Montanide), and mixed by POLYTRON PT3100 (kinematic model) at 2,000 rpm for 1 hour. For group 2, each 100 μg of the immunogen peptides (tetrameric MAP peptide with SEQ ID NO 1 connected to the C terminus of GnRH B epitope) is solubilized in 200 μl PBS, emulsified in 200 μl ISA 206 (Montanide), and mixed by POLYTRON PT3100 (kinematic model) at 2,000 rpm for 1 hour. For the first control group (group 3), a carrier protein PEK8 (Pseudomonad Exotoxin with additional C-terminal 8 Lysine amino acids) chemically conjugated with GnRH (PEK8G) was used. For the secod control group (group 4), adjuvant plus PBS was used. For the third control group (group 5), no injections were giving to the castrated rats. Third, on week 0, week 2, and week 4, each of the rats in the first and second groups was then inoculated with the respective peptide immunogen at a dosage of 100 μg/400 μl subcutaneously. Rats in group 3 were injected with 400 μl of adjuvant plus PEK8G. Rats in one control group (group 4) were injected with 400 μl of adjuvant plus PBS. Castrated rats in another control group were not given any injection. Blood was collected on 2 wpsb, i.e. 2 weeks post second boost injection. The blood was centrifuged at 5,000 r.p.m. for 10 minutes and sera were stored at −20° C. in a freezer.

C. Immunogenicity Determination. Serum samples were assayed for antibodies against various peptides of G4 (tetrameric MAP form of GnRH), (T1)⁸, (sT1)⁸, and (tT1)⁸. ELISA assays were performed using 96-well ELISA plates (Nalge nunc). The various peptide antigens were adsorbed to the plates, at a concentration of 0.5 μg/well in 100 μl/well of bicarbonate coating buffer (1.378 g Na₂CO₃, 2.94 g NaHCO₃ in 1L ddH₂O) and incubated overnight at 4° C. Then, the coating buffer was discarded and, ELISA plates were washed three times with wash buffer (0.5 ml Tween-20 in one litter of 1×PBS). Then the plates were blocked with 5% BSA at 100 μl per well and incubated over night at 4° C. Blocking solution was discarded and the plates were stored at −20° C. until use. Test sera were diluted 1:100× in 5% BSA and placed at 100 μl per well. Test sera were allowed to react at room temperature for 2 hours. Test sera dilutions were then discarded and plates washed three times with wash buffer. Then goat-anti-rat IgG (Sigma, whole molecular, A-8438, Lot 95H8940, Alkaline phosphatase conjugate) at 1:10000× dilution dilution were placed at 100 μl per well and allowed to react at room temperature for 2 hours. Sera dilution was then discarded, and the plates were washed three times with wash buffer. 100 μl/well of color developing buffer (15 mg pNPP (3 tablets of product No. 34047 of Pierce) in 15 ml of 10 mM Diethanolamine buffer (PH 9.5)) were added to the plates and allowed to develop for half an hour at 37° C. The absorbances, optical density, were measured at 405 nm.

D. Immunogen Bioefficacy Determination. Testosterone levels of the four groups of rats were determined on 2 wpb, i.e. 2 weeks post boost injection, with the Ciba Corning Automated Chemiluminescence (ACS™) Testosterone assay kit. The ACS Testosterone assay measures testosterone concentration up to 1500 ng/dL with a minimum detectable concentration of 10 ng/dL (=0.347 nmol/L).

E. Results. As shown in FIG. 2, on 2 wpb, both peptide immunogens T1G and GT1 raised antibody response against G4. In terms of the fine specificity of antibodies raised, T1G also produced high antibody response to (T1)⁸ and its derivatives (sT1)⁸ and (tT1)⁸ than GT1, see FIG. 2A. With respect to testosterone reduction, all four rats injected with T1G has a reduced testosterone level at the responder level, see FIG. 2B.

### EXAMPLE 3

A. Peptide Synthesis. The peptide immunogen comprising Th epitope of SEQ ID NO 1 connected to the N terminus of GnRH B epitope is synthesized in tetrameric MAP form. The synthesis of tetrameric MAP peptides were accomplished manually by a stepwise solid-phase procedure with Fmoc strategy on

[Fmoc-Lys(Fmoc)]2-Lys-βAla-Wang resin (0.77 mmol/g, ACT, Louisville, Ky., USA). The coupling of Fmoc amino acids was performed in N-methylpyrrolidone using the dicyclohexylcarbodiimide/hydroxybenzotriazole procedure. After completion of the MAP peptide synthesis, deprotection and cleavage from resin support is accomplished by treatment with trifluoroacetic acid. The final products were characterized by reverse phase high performance liquid chromatography and MALDI TOF Mass Spectrum.

B. Immunization Protocol. Five juvenile beagles of 3.5 months old were used. They were bred under specific pathogen free conditions and transferred to a conventional animal house for the experiment. All aspects of the work including housing, experimentation and disposal of animals were performed in general according to the International Guiding Principles for Biomedical Research Involving Animals (CIOMS Publication No. ISBN 92 90360194, 1985). First, the beagles were weighed. The average weight of beagles at 3.5 months old is 5.83 kg. Second, for test group 1 (beagle number B83, B87), 40 μg of the immunogen peptides (tetrameric MAP peptide with SEQ ID NO 1 connected to the N terminus of GnRH B epitope) is solubilized in 100 μl PBS, emulsified with an equal volume of adjuvant ISA 206 (Montanide), and mixed by POLYTRON PT3100 (kinematic model) at 2,000 rpm for 1 hour. For test group 2 (beagle number B84, B85, B86), 160 μg of the immunogen peptides (tetrameric MAP peptide with SEQ ID NO 1 connected to the N terminus of GnRH B epitope) is solubilized in 200 μl PBS, emulsified with an equal volume of adjuvant ISA 206 (Montanide), and mixed by POLYTRON PT3100 (kinematic model) at 2,000 rpm for 1 hour. Third, on month 0, the beagles in test group 1 and 2 test groups were then inoculated subcutaneously with the T1G peptide immunogen at the dosage of 40 μg/200 μl and 160 μg/400 μl, respectively. Boost injections with same inoculations were given subcutaneously on month 3. Fourth, blood was collected from the median cubital vein each month. The blood was centrifuged at 5,000 r.p.m. for 10 minutes and sera were stored at −20° C. in a freezer.

C. Immunogenicity Determination. Serum samples were assayed for antibodies against G4. ELISA assays were performed using 96-well ELISA plates (Nalge nunc). The various peptide antigens were adsorbed to the plates, at a concentration of 0.5 μg/well in 100 μl/well of bicarbonate coating buffer (1.378 g Na₂CO₃, 2.94 g NaHCO₃ in 1L ddH₂O) and incubated overnight at 4° C. Then, the coating buffer was discarded and, ELISA plates were washed three times with wash buffer (0.5 ml Tween-20 in one litter of 1×PBS). Then the plates were blocked with 5% BSA at 100 μl per well and incubated overnight at 4° C. Blocking solution was discarded and the plates were stored at −20° C. until use. Test serawere diluted 1:100× in 5% BSA and placed at 100 μl per well. Test sera were allowed to react at room temperature for 2 hours. Test sera dilutions were then discarded and plates washed three times with wash buffer. Then rabbit anti-dog IgG (whole molecule) conjugated with alkaline phosphatase (Sigma, A 0793) at 1:4000× dilution were placed at 100 μl per well and allowed to react at room temperature for 2 hours. The antibody dilution was then discarded, and the plates were washed three times with wash buffer. 100 μl/well of color developing buffer (15 mg pNPP (3 tablets of product No. 34047 of Pierce) in 15 ml of 10 mM Diethanolamine buffer (PH 9.5)) were added to the plates and allowed to develop for half an hour at 37° C. The absorbances, optical density, were measured at 405 nm.

D. Immunogen Bioefficacy Determination. Testosterone levels of the two groups of bseagle were determined on 2 mpi (months post primary inoculation), 3 mpi, 4 mpi, and 5.5 mpi with the Ciba Corning Automated Chemiluminescence (ACS™) Testosterone assay kit. The ACS Testosterone assay measures testosterone concentration up to 1500 ng/dL with a minimum detectable concentration of 10 ng/dL (=0.347 nmol/L). Serum testosterone levels below 10 ng/dL was considered “castration” levels, however, below 57.6 ng/dL (2 nmo/L) was considered as a responder of the immunocontraception vaccine studied.

E. Results. As shown in FIG. 3, T1G produced the highest antibody response against G4 at 4 month post primary inoculation at the dosage of 160 μg. T1G also significantly reduced the testosterone level in beagles numbered B85 and B86 at a reading of 9 and 7 ng/dL respectively at 4 months post primary inoculation.

### EXAMPLE 4

A. Peptide Synthesis. The peptide immunogen comprising Th epitope of SEQ ID NO 2 connected to GnRH is synthesized in tetrameric MAP form. The synthesis of tetrameric MAP peptides were accomplished manually by a stepwise solid-phase procedure with Fmoc strategy on [Fmoc-Lys(Fmoc)]2-Lys-βAla-Wang resin (0.77 mmol/g, ACT, Louisville, Ky., USA). The coupling of Fmoc amino acids was performed in N-methylpyrrolidone using the dicyclohexylcarbodiimide/hydroxybenzotriazole procedure. After completion of the MAP peptide synthesis, deprotection and cleavage from resin support is accomplished by treatment with trifluoroacetic acid. The final products were characterized by reverse phase high performance liquid chromatography and MALDI TOF Mass Spectrum.

B. Immunization Protocol. Two groups of five male BALB/c mice were used at 4-5 weeks old, with one group as a control group. They were bred under specific pathogen free conditions and transferred to a conventional animal house for the experiment. All aspects of the work including housing, experimentation and disposal of animals were performed in general according to the International Guiding Principles for Biomedical Research Involving Animals (CIOMS Publication No. ISBN 92 90360194, 1985). The mice were weighed. The average weight of male BALB/c mice at 4-5 weeks old is 35 grams. Second, for the test group, 50 μg of the immunogen peptides (tetrameric MAP peptide with SEQ ID NO 2 connected to the N terminus of GnRH B epitope) is solubilized in 100 μp PBS, emulsified with an equal volume of adjuvant ISA 206 (Montanide), and mixed by POLYTRON PT3100 (kinematic model) at 2,000 rpm for 1 hour. For the control group, adjuvant plus PBS was used. Third, on week 0, each of the mice in the test group was then inoculated with the respective peptide immunogen at a dosage of 50 μg/200 μl subcutaneously. Mice in the control group were injected with 200 μl of adjuvant plus PBS. Boost injections with same inoculations were given subcutaneously on weeks, 2, and 4. Fourth, blood was collected by retroorbital plexus puncture on the 6th, 8th, and 10th week for ELISA assay. The blood was centrifuged at 5,000 r.p.m. for 10 minutes and sera were stored at −20° C. in a freezer.

C. Immunogenicity Determination. Serum samples were assayed for antibodies against various peptides of G4, (T1)⁸, (sT1)⁸, and (tT1)⁸ by ELISA. ELISA assays were performed using 96-well ELISA plates (Nalge nunc). The various peptide antigens were adsorbed to the plates, at a concentration of 0.5 μg/well in 100 μl/well of bicarbonate coating buffer (1.378 g Na₂CO₃, 2.94 g NaHCO₃ in 1 L ddH₂O) and incubated overnight at 4° C. Then, the coating buffer was discarded and, ELISA plates were washed three times with wash buffer (0.5 ml Tween-20 in one litter of 1×PBS). Then the plates were blocked with 5% BSA at 100 μl per well and incubated overnight at 4° C. Blocking solution was discarded and the plates were stored at −20° C. until use. Test sera were diluted 1:100× in 5% BSA and placed at 100 μl per well. Test sera were allowed to react at room temperature for 2 hours. Test sera dilutions were then discarded and plates washed three times with buffer. Then goat-anti-mouse IgG (Sigma, Fab specific, A-1293, Lot 28H4859, Alkaline phosphatase conjugate) at 1:5000× dilution were placed at 100 μl per well and allowed to react at room temperature for 2 hours. The antibody dilution was then discarded, and the plates were washed three times with wash buffer. 100 μl/well of color developing buffer (15 mg pNPP (3 tablets of product No. 34047 of Pierce) in 15 ml of 10 mM Diethanolamine buffer (PH 9.5)) were added to the plates and allowed to develop for half an hour at 37° C. The absorbances, optical density, were measured at 405 nm.

D. Immunogen Bioefficacy Determination. Testosterone levels of the two groups of mice were determined on 6 wpi, 8 wpi, and 10 wpi with the Ciba Corning Automated Chemiluminescence (ACS™) Testosterone assay kit. The ACS Testosterone assay measures testosterone concentration up to 1500 ng/dL with a minimum detectable concentration of 10 ng/dL (=0.347 nmol/L). Serum testosterone levels below 10 ng/dL was considered “castration” levels, however, below 57.6 ng/dL (2 nmo/L) was considered as a responder of the immunocontraception vaccine studied.

E. Results. As shown in FIG. 4, sT1G produced the highest antibody response against G4 on week 10 post primary inoculation. In terms of the fine specificity of antibodies raised, sT1G also produced antibody response to (T1)⁸ and its derivatives (sT1)⁸ and (tT1)⁸ with the stronger response to (tT1)⁸. With respect to testosterone reduction, sT1G did not show significant result in reducing testosterone level in male mice.

### EXAMPLE 5

A. Peptide Synthesis. The peptide immunogen comprising Th epitope of SEQ ID NO 3 connected to GnRH is synthesized in tetrameric MAP form. The synthesis of tetrameric MAP peptides were accomplished manually by a stepwise solid-phase procedure with Fmoc strategy on [Fmoc-Lys(Fmoc)]2-Lys-βAla-Wang resin (0.77 mmol/g, ACT, Louisville, Ky., USA). The coupling of Fmoc amino acids was performed in N-methylpyrrolidone using the dicyclohexylcarbodiimide/hydroxybenzotriazole procedure. After completion of the MAP peptide synthesis, deprotection and cleavage from resin support is accomplished by treatment with trifluoroacetic acid. The final products were characterized by reverse phase high performance liquid chromatography and MALDI TOF Mass Spectrum.

B. Immunization Protocol. Two groups of five male BALB/c mice were used at 4-5 weeks old, with one group as a control group. They were bred under specific pathogen free conditions and transferred to a conventional animal house for the experiment. All aspects of the work including housing, experimentation and disposal of animals were performed in general according to the International Guiding Principles for Biomedical Research Involving Animals (CIOMS Publication No. ISBN 92 90360194, 1985). First, the mice were weighed. The average weight of male BALB/c mice at 4-5 weeks old is 35 grams. Second, for the test group, 50 μg of the immunogen peptides (tetrameric MAP peptide with SEQ ID NO 3 connected to the N terminus of GnRH B epitope) is solubilized in 100 μl PBS, emulsified with an equal volume of adjuvant ISA 206 (Montanide), and mixed by POLYTRON PT3100 (kinematic model) at 2,000 rpm for 1 hour. For the control group, adjuvant plus PBS was used. Third, on week 0, each of the mice in the test group was then inoculated with the respective peptide immunogen at a dosage of 50 μg/200 μl subcutaneously. Mice in the control group were injected with 200 μl of adjuvant plus PBS. Boost injections with same inoculations were given subcutaneously on weeks 2, and 4. Fourth, blood was collected by retroorbital plexus puncture on the 6th, 8th, and 10th week for ELISA assay. The blood was centrifuged at 5,000 r.p.m. for 10 minutes and sera were stored at −20° C. in a freezer.

C. Immunogenicity Determination. Serum samples were assayed for antibodies against various peptides of G4, (T 1)₈, (sT1)₈, and (tT1)₈ by ELISA. ELISA assays were performed using 96-well ELISA plates (Nalge nunc). The various peptide antigens were adsorbed to the plates, at a concentration of 0.5 μg/well in 100 μl/well of bicarbonate coating buffer (1.378 g Na₂CO₃, 2.94 g NaHCO₃ in 1 L ddH₂O) and incubated overnight at 4° C. Then, the coating buffer was discarded and, ELISA plates were washed three times with wash buffer (0.5 ml Tween-20 in one litter of 1×PBS). Then the plates were blocked with 5% BSA at 100 μl per well and incubated overnight at 4° C. Blocking solution was discarded and the plates were stored at −20° C. until use. Test sera were diluted 1:100× in 5% BSA and placed at 100 μl per well. Test sera were allowed to react at room temperature for 2 hours. Test sera dilutions were then discarded and plates washed three times with wash buffer. Then goat-anti-mouse IgG (Sigma, Fab specific, A-1293, Lot 28H4859, Alkaline phosphatase conjugate) at 1:5000× dilution were placed at 100 μl per well and allowed to react at room temperature for 2 hours. The antibody dilution was then discarded, and the plates were washed three times with wash buffer. 100 μl/well of color developing buffer (15 mg pNPP (3 tablets of product No.34047 of Pierce) in 15 ml of 10 mM Diethanolamine buffer (PH 9.5)) were added to the plates and allowed to develop for half an hour at 37° C. The absorbances, optical density, were measured at 405 nm.

D. Immunogen Bioefficacy Determination. Testosterone levels of the two groups of mice were determined on 6 wpi, 8 wpi, and 10 wpi with the Ciba Corning Automated Chemiluminescence (ACS™) Testosterone assay kit. The ACS Testosterone assay measures testosterone concentration up to 1500 ng/dL with a minimum detectable concentration of 10 ng/dL (=0.347 nmol/L). Serum testosterone levels below 10 ng/dL was considered “castration” levels, however, below 57.6 ng/dL (2 nmo/L) was considered as a responder of the immunocontraception vaccine studied.

E. Results. As shown in FIG. 5, tT1G produced the highest antibody response against G4 on week 6 post primary inoculation. In terms of the fine specificity of antibodies raised, sT1G also produced antibody response to (T1)⁸ and its derivatives (sT1)⁸ and (tT1)⁸ with the stronger response to (tT1)⁸. With respect to testosterone reduction, tT1G significantly reduced testosterone level in male mice on week 10 post primary inoculation with a reading at 34 ng/dL.

### EXAMPLE 6

A. Peptide Synthesis. The peptide immunogens comprising Th epitope of SEQ ID NO 4 connected to the N terminus and C terminus of the GnRH B epitopeis are synthesized in tetrameric MAP form. The synthesis of tetrameric MAP peptides were accomplished manually by a stepwise solid-phase procedure with Fmoc strategy on [Fmoc-Lys(Fmoc)]2-Lys-βAla-Wang resin (0.77 mmol/g, ACT, Louisville, Ky., USA). The coupling of Fmoc amino acids was performed in N-methylpyrrolidone using the dicyclohexylcarbodiimide/hydroxybenzotriazole procedure. After completion of the MAP peptide synthesis, deprotection and cleavage from resin support is accomplished by treatment with trifluoroacetic acid. The final products were characterized by reverse phase high performance liquid chromatography and MALDI TOF Mass Spectrum.

B. Immunization Protocol. Three groups of five male BALB/c mice were used at 4-5 weeks old, with one group as a control group. They were bred under specific pathogen free conditions and transferred to a conventional animal house for the experiment. All aspects of the work including housing, experimentation and disposal of animals were performed in general according to the International Guiding Principles for Biomedical Research Involving Animals (CIOMS Publication No. ISBN 92 90360194, 1985). First, the mice were weighed. The average weight of male BALB/c mice at 4-5 weeks old is 35 grams. Second, for group 1, 50 μg of the immunogen peptides (tetrameric MAP peptide with SEQ ID NO 4 connected to the N terminus of GnRH B epitope) is solubilized in 100 μl PBS, emulsified with an equal volume of adjuvant ISA 206 (Montanide), and mixed by POLYTRON PT3100 (kinematic model) at 2,000 rpm for 1 hour. For group 2, 50 μg of the immunogen peptides (tetrameric MAP peptide with SEQ ID NO 4 connected to the C terminus of GnRH B epitope) is solubilized in 100 μl PBS, emulsified in 100 μl ISA 206 (Montanide), and mixed by POLYTRON PT3100 (kinematic model) at 2,000 rpm for 1 hour. For the control group, adjuvant plus PBS was used. Third, on week 0, each of the mice in the first and second groups was then inoculated with the respective peptide immunogen at a dosage of 50 μg/200 μl subcutaneously. Mice in the control group were injected with 200 μl of adjuvant plus PBS. Boost injections with same inoculations were given subcutaneously on weeks 2, and 4. Fourth, blood was collected by retroorbital plexus puncture on the 6th, 8th, and 10th week for ELISA assay. The blood was centrifuged at 5,000 r.p.m. for 10 minutes and sera were stored at −20° C. in a freezer.

C. Immunogenicity Determination. Serum samples were assayed for antibodies against various peptides of G4, (T1)⁸, (sT1)⁸, and (tT1)⁸ by ELISA. ELISA assays were performed using 96-well ELISA plates (Nalge nunc). The various peptide antigens were adsorbed to the plates, at a concentration of 0.5 μg/well in 100 μl/well of bicarbonate coating buffer (1.378 g Na₂CO₃, 2.94 g NaHCO₃ in 1 L ddH₂O) and incubated overnight at 4° C. Then, the coating buffer was discarded and, ELISA plates were washed three times with wash buffer (0.5 ml Tween-20 in one litter of 1×PBS). Then the plates were blocked with 5% BSA at 100 μl per well and incubated overnight at 4° C. Blocking solution was discarded and the plates were stored at −20° C. until use. Test sera were diluted 1:100× in 5% BSA and placed at 100 μl per well. Test sera were allowed to react at room temperature for 2 hours. Test sera dilutions were then discarded and plates washed three times with wash buffer. Then goat-anti-mouse IgG (Sigma, Fab specific, A-1293, Lot 28H4859, Alkaline phosphatase conjugate) at 1:5000 × dilution were placed at 100 μl per well and allowed to react at room temperature for 2 hours. The antibody dilution was then discarded, and the plates were washed three times with wash buffer. 100 μl/well of color developing buffer (15 mg pNPP (3 tablets of product No. 34047 of Pierce) in 15 ml of 10 mM Diethanolamine buffer (PH 9.5)) were added to the plates and allowed to develop for half an hour at 37° C. The absorbances, optical density, were measured at 405 nm.

D. Immunogen Bioefficacy Determination. Testosterone levels of the three groups of mice were determined on 6 wpi, 8 wpi, and 10 wpi with the Ciba Corning Automated Chemiluminescence (ACS™) Testosterone assay kit. The ACS Testosterone assay measures testosterone concentration up to 1500 ng/dL with a minimum detectable concentration of 10 ng/dL (=0.347 nmol/L). Serum testosterone levels below 10 ng/dL was considered “castration” levels, however, below 57.6 ng/dL (2 nmo/L) was considered as a responder of the immunocontraception vaccine studied.

E. Results. As shown in FIG. 6, on week 10 post primary inoculation, both peptide immunogens PG and GP raised high antibody response against G4, however, PG has a stronger antibody response against G4. With respect to testosterone reduction, both PG and GP did not have any significant result in reducing testosterone level in male mice.

What is claimed is:

1. A peptide immunogen, comprising the formula:
[(Th)-(B)ₒ-(Target Antigenic Site)]₄K₂K-βA
or
[(Th)-(B)ₒ-(Target Antigenic Site)]₈K₄K₂K-βA
or
[(Target Antigenic Site)-(B)ₒ-(Th)]₄K₂K-βA
or
[(Target Antigenic Site)-(B)ₒ-(Th)]₈K₄K₂K-βA
wherein:
Th is a helper T cell epitope comprising a sequence at least 50% identical to SEQ ID NO:1;
B is a spacer;
Target Antigenic Site is a B epitope;
K is a bifunctional unit;
βA is beta Alanine;
₀ is an integer from 0 to 1.

2. The peptide immunogen of claim 1, wherein the spacer comprises three or fewer Glycine residues.

3. The peptide immunogen of claim 1, wherein said B epitope is GnRH.

4. The peptide immunogen of claim 1, wherein said bifunctional unit comprises an amino acid selected from the group comprising of cysteine, lysine, aspartic acid, glutamic acid, and ornithine.

5. A composition, comprising an immunologically effective amount of the peptide immunogen of claim 1.

6. The composition of claim 5, further comprising a pharmaceutically acceptable carrier.

7. The composition according to claim 5, wherein said immunologically effective amount of said peptide immunogen is between about 0.005 mg to 1.5 mg per kilogram body weight per dose.

8. A method of generating antibodies specific for the peptide immunogen of claim 1, comprising the step of introducing into an animal a composition comprising the peptide immunogen of claim 1.

9. The method of claim 8, wherein said B epitope is from a self molecule.

10. An antibody that binds specifically to the peptide immunogen of claim 1.

11. An antibody that selectively binds to an epitope that is within the sequence of SEQ ID NO5.

12. An antibody that is specific for Gonadotropin and reduces serum testosterone level to less than 57.6 ng/dL when produced by administering the peptide immunogen of claim 1 to mammalian animals at a dosage of about 0.005 mg/kg to about 1.43 mg/kg.

13. A method of reducing the serum testosterone level in an animal, comprising administering to the animal a composition comprising the antibody of claim 10, 11, or 12.

14. A peptide immunogen, comprising the formula:
[(Th)-(B)ₒ-(Target Antigenic Site)]₄K₂K-βA
or
[(Th)-(B)ₒ-(Target Antigenic Site)]₈K₄K₂K-βA
or
[(Target Antigenic Site)-(B)ₒ-(Th)]₄K₂K-βA
or
[(Target Antigenic Site)-(B)ₒ-(Th)]₈K₄K₂K-βA
wherein:
Th is a helper T cell epitope comprising a sequence at least 50% identical to SEQ ID NO. 2;
B is a spacer;
Target Antigenic Site is a B epitope;
K is a bifunctional unit;
βA is beta Alanine;
₀ is from0 to 1.

15. The peptide immunogen of claim 14, wherein the spacer comprises three or fewer Glycine resideues.

16. The peptide immunogen of claim 14, wherein said B epitope is GnRH.

17. The peptide immunogen of claim 14, wherein said bifunctioanl unit comprises an amino acid selected from the group comprising of cysteine, lysine, aspartic acid, glutamic acid, and ornithine.

18. A composition, comprising an immunologically effective amount of the peptide immunogen of claim 14.

19. The composition of claim 18, further comprising a pharmaceutically acceptable carrier.

20. The composition according to claim 18, wherein said immunologically effective amount of said peptide immunogen is between about 0.005 mg to 1.5 mg per kilogram body weight per dose.

21. A method of generating antibodies specific for the peptide immunogen of claim 14, comprising the step of introducing into an animal a composition comprising the peptide immunogen of claim 14.

22. The method of claim 21, wherein said B epitope is from a self molecule.

23. An antibody that binds specifically to the peptide immunogen of claim 14.

24. A peptide immunogen, comprising the formula:
[(Th)-(B)ₒ-(Target Antigenic Site)]₄K₂K-βA
or
[(Th)-(B)ₒ-(Target Antigenic Site)]₈K₄K₂K-βA
or
[(Target Antigenic Site)-(B)ₒ-(Th)]₄K₂K-βA
or
[(Target Antigenic Site)-(B)ₒ-(Th)]₈K₄K₂K-βA
wherein:
Th is a helper T cell epitope comprising a sequence at least 50% identical to SEQ ID NO. 3;
B is a spacer;
Target Antigenic Site is a B epitope;
K is a bifunctional unit;
βA is beta Alanine;
₀ is from0to 1.

25. The peptide immunogen of claim 24, wherein the spacer comprises three or fewer Glycine residues.

26. The peptide immunogen of claim 24, wherein said B epitope is GnRH.

27. The peptide immunogen of claim 24, wherein said bifunctional unit comprises an amino acid selected from the group comprising of cysteine, lysine, aspartic acid, glutamic acid, and ornithine.

28. A composition, comprising an immunologically effective amount of the peptide immunogen of claim 24.

29. The composition of claim 28, further comprising a pharmaceutically acceptable carrier.

30. The composition according to claim 28, wherein said immunologically effective amount of said peptide immunogen is between about 0.005 mg to 1.5 mg per kilogram body weight per dose.

31. A method of generating antibodies specific for the peptide immunogen of claim 24, comprising the step of introducing into an animal a composition comprising the peptide immunogen of claim 24.

32. The method of claim 31, wherein said B epitope is from a self molecule.

33. An antibody that binds specifically to the peptide immunogen of claim 24.

34. An antibody that is specific for Gonadotropin and reduces serum testosterone level to less than 57.6 ng/dL when produced by administering peptide immunogen of claim 24 to mammalian animals at a dosage of about 1.43 mg/kg.

35. A method of reducing the serum testosterone level in an animal, comprising administering to the animal a composition comprising the antibody of claim 33 or 34.

36. A peptide immunogen, comprising the formula:
[(Th)-(B)ₒ-(Target Antigenic Site)]₄K₂K-βA
or
[(Th)-(B)ₒ-(Target Antigenic Site)]₈K₄K₂K-βA
or
[(Target Antigenic Site)-(B)ₒ-(Th)]₄K₂K-βA
or
[(Target Antigenic Site)-(B)ₒ-(Th)]₈K₄K₂K-βA
wherein:
Th is a helper T cell epitope comprising a sequence at least 50% identical to SEQ ID NO. 4;
B is a spacer;
Target Antigenic Site is a B epitope;
K is a bifunctional unit;
βA is beta Alanine;
₀ is from 0 to 1.

37. The peptide immunogen of claim 36, wherein the spacer comprises three or fewer Glycine residues.

38. The peptide immunogen of claim 37, wherein said B epitope is GnRH.

39. The peptide immunogen of claim 36, wherein said bifunctional unit comprises an amino acid selected from the group comprising of cysteine, lysine, aspartic acid, glutamic acid, and ornithine.

40. A composition, comprising an immunologically effective amount of the. peptide immunogen of claim 36.

41. The composition of claim 40, further comprising a pharmaceutically acceptable carrier.

42. The composition according to claim 40, wherein said immunologically effective amount of said peptide immunogen is between about 0.005 mg to 1.5 mg per kilogram body weight per dose.

43. A method of generating antibodies specific for the peptide immunogen of claim 36, comprising the step of introducing into an animal a composition comprising the peptide immunogen of claim 36.

44. The method of claim 43, wherein said B epitope is from a self molecule.

45. An antibody that binds specifically to the peptide immunogen of claim 36.


SEQUENCE LISTING (5 sequences)

SEQ ID NO: 1 - protein, 13 aa, Influenza virus
  PEPTIDE (1)..(13)
PKYVKQNTLKLAT

SEQ ID NO: 2 - protein, 10 aa, Influenza virus
  PEPTIDE (1)..(10)
KYVKQNTLKL

SEQ ID NO: 3 - protein, 8 aa, Influenza virus
  PEPTIDE (1)..(8)
PKYVKQNT

SEQ ID NO: 4 - protein, 13 aa, Enterovirous Poliovirus
  PEPTIDE (1)..(13)
KLFAVWKITYKDT

SEQ ID NO: 5 - protein, 10 aa, mammalian
  PEPTIDE (1)..(10)
EHWSYGLRPG